(19) United States
(12) Patent Application Publication
(10) Pub. No.: US 2004/0191301 A1
(43) Pub. Date: Sep. 30, 2004

(54) Transdermal device having a phase change material

(76) Inventor: Albert Philip Van Duren, Chaska, MN (US)

Correspondence Address: INCAPLAW, TERRANCE A. MEADOR, 1050 ROSECRANS STREET, SUITE K, SAN DIEGO, CA, 92106, US

(21) Appl. No.: 10/402,023

(22) Filed: Mar. 27, 2003

(51) Int. Cl. A61K 9/70

(52) U.S. Cl. 424/449

(57) ABSTRACT

A transdermal device contains a phase change material and another material. The transdermal device may also contain a drug, a pharmaceutical, or a medicament mixed with or disposed near the phase change material. The phase change material transitions from solid to liquid form when heated. The other material is disposed, suspended, or held in the phase change material when the phase change material is in its solid form. When heat is applied to the transdermal device, the phase change material changes to a liquid state and the material that is held or suspended in the phase change material is released. Advantageously, such materials may include substances such as pharmaceuticals that boost or supplement the effect produced by a drug delivered transdermally by the device, or drug antagonists.

### FIELD OF THE INVENTION

This invention relates to devices put on the skin to apply pharmacological agents, medicaments, or drugs, including analgesic agents, transdermally, that is, through the skin. More particularly, the invention relates to a transdermal device incorporating a phase change material that changes properties and characteristics of the transdermal device with the addition of heat.

### BACKGROUND OF THE INVENTION

The term “transdermal” refers to introduction or delivery of healing or treatment materials such as pharmacological agents, medicaments, or drugs into a human or animal body through the skin. Transdermal delivery of such materials provides many advantages. For example, such a mode of delivery is comfortable, convenient and noninvasive. With transdermal delivery, the variable rates of absorption and metabolism encountered in oral treatment are avoided, and other inherent inconveniences, such as gastrointestinal irritation and the like, are eliminated. Transdermal delivery also makes possible a high degree of control over blood concentrations of any particular agent, medicament, or drug. Over the years the types of materials that can be delivered with a transdermal patch have increased tremendously. For example, many analgesic drug compounds such as fentanyl, sufentanil, alfentanyl, remifentanil, and morphine are administered with a transdermal device. Transdermal devices include, without limitation, bandages, patches, plasters, and other equivalent apparatus.

Another method of drug delivery through the skin is called iontophoresis. By applying a low-level electrical current to a similarly charged drug solution, iontophoresis repels the drug ions through the skin to the underlying tissue. In contrast to passive transdermal patch drug delivery, iontophoresis is an active method that allows the delivery of ionic drugs that are not effectively absorbed through the skin.

A representative transdermal device, described in U.S. Pat. No. 4,286,592, is a bandage for delivering drugs to the skin. The bandage consists of a drug reservoir layer sandwiched between an impermeable backing layer and a permeable contact adhesive layer. A drug dispersed in a carrier is disposed in the drug reservoir layer. The drug diffuses through the contact adhesive layer at a rate that is determined by factors common to the drug, the carrier, and the contact adhesive material.

Currently, transdermal patches are designed to deliver medication slowly through the skin over a long period of time, usually 48 to 72 hours. It is important to know that it may take considerable time, sometimes as long as 12 hours, before the device has reached its full treatment capacity. Depending upon the condition that is being treated, such long-term action can decrease the clinical effectiveness of the treatment. For example, in the treatment of chronic pain, one of the drawbacks with the slow delivery of medication by way of a transdermal patch is that many people experience intermittent flares of pain. Severe flares are called breakthrough pain. The characteristics of breakthrough pain, including the duration of a breakthrough episode, vary from person to person. Generally, breakthrough pain happens quickly, and varies in duration. It may occur unexpectedly and for no obvious reason, or it may be triggered by a specific activity, like coughing, moving, or going to the bathroom.

Ideally, medication for breakthrough pain should be easily administered and work rapidly. Most people prefer oral medications (taken by mouth), but these are frequently slow to take effect. Further, orally-administered medications are known to cause difficulty in swallowing, nausea or other gastrointestinal problems. Pain medication may also be given by injection, either manually or through a patient-controlled analgesia device (PCA), or through an intravenous tube. But these modes are usually administered by trained personnel and may not compatible with home care. Accordingly, administration by transdermal means is increasingly favored.

The transdermal administration of some drugs is advantageously assisted by the addition of heat. For example, U.S. Pat. No. 6,261,595 describes a transdermal patch with an attached pocket for receiving a heating device. Distinct advantages enjoyed with the application of heat include a decrease in the time required for the transdermal patch to reach steady-state serum concentrations, and the ability to change the serum concentration of the drug being delivered for a short period of time in response to the changes in the magnitude of applied heat. Of course, increasing the rate at which medication is administered from a transdermal patch may mean more frequent changing of the patch.

There is sometimes a social price paid for the convenience of administering medication by transdermal means. In this regard, transdermal devices are frequently stolen and then processed to extract narcotic drugs for unauthorized use. A drug may be easily obtained from a transdermal patch by boiling the patch in water. The diluted drug may then be concentrated, aggregated with a larger supply and ingested. One method to combat such abuse is disclosed in U.S. Pat. No. 5,149,538 in which an analgesic antagonist is added to a transdermal patch containing an analgesic drug. Initially, the antagonist is separated from the analgesic drug by a barrier that is soluble in water, alcohol or an organic solvent. In use, if the transdermal patch is immersed in water, the barrier dissolves, releasing the antagonist to mix with, and neutralize, the analgesic drug. One disadvantage of this design is the soluble barrier, which may release the antagonist prematurely if the patch gets wetted accidentally, or is disposed in a humid environment, such as when a user showers or bathes.

It would be advantageous to elaborate or enhance the ability of a transdermal patch to alter the characteristics of drug delivery with the addition of heat, by, for example, sharply increasing or decreasing the rate of drug administration to a patient beyond that which is achievable presently. For example, treatment of breakthrough pain can be enhanced by a steeply accelerating rate of delivery in response to an increase in heat.

It also would be desirable to defeat abuse of a transdermal patch in response to elevation of the temperature of the patch beyond a threshold that approaches the temperature of water heated to, or near, boiling.

### SUMMARY OF INVENTION

The present invention is directed to a transdermal device that contains a phase change material and another material. The transdermal device may also contain a drug, a pharmaceutical, or a medicament mixed with or disposed near the phase change material. The term “phase change material” denotes a substance that changes state in response to heat. In particular, a phase change material is one that transitions from solid to liquid form with the addition of heat. The other material is disposed, suspended, or held in the phase change material when the phase change material is in its solid form. When heat is applied to the transdermal device the phase change material changes to a liquid state and the material that is held or suspended in the phase change material is released. Advantageously, such materials may include substances such as pharmaceuticals, drug antagonists or agonists.

### BRIEF DESCRIPTION OF THE DRAWINGS

FIG. 1 is a sectional view showing one embodiment of the invention;

FIG. 2 is a sectional view showing an embodiment similar to FIG. 1 using an alternate adhesive layer;

FIG. 3 is a sectional view showing another embodiment of the invention; and

FIGS. 4-6 are sectional views showing more embodiments of the reservoir and phase change material layer.

### DETAILED DESCRIPTION OF THE EMBODIMENTS

In this description, a transdermal device is presented and discussed. For convenience, the device is referred to as a “transdermal patch” or simply as a “patch”; however, use of these terms is not intended, and should not be taken, to exclude from the scope of this invention other devices that are designed to be placed on the skin to enable the transdermal administration of a drug, a pharmaceutical, or a medicament.

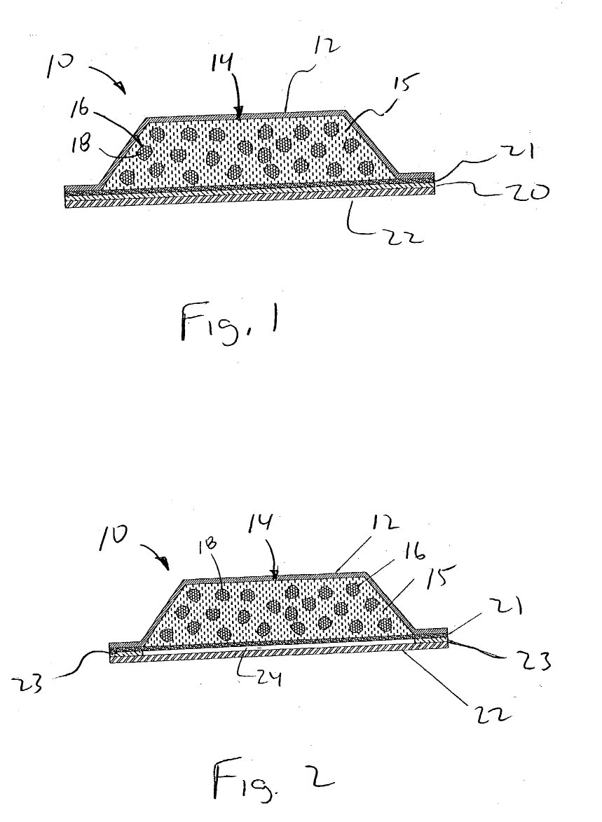

FIG. 1 illustrates one embodiment of this invention. A transdermal patch 10 is comprised of a backing layer 12, a reservoir 14 containing a drug 15 and a phase change material preferably having a particulate form including particles 16. The particles and the drug are mixed so that the particles are dispersed throughout the drug 15. A material 18 is held or suspended in the phase change material particles 16 in their solid state. In this condition, the material 18 is immobilized and is substantially out of contact with the drug 15. This is the state that is illustrated in FIG. 1. When the phase change material transitions from solid to liquid form, the material 18 emerges, or is released, from the phase change material at which time it becomes mobile and comes into contact with the drug 15. This is not shown, but is inherent in, and readily conceivable from FIG. 1. The patch 10 may also have an adhesive layer 20 connected to the backing layer 14 directly, or through intermediate structure not shown in FIG. 1. The adhesive layer 20 may also contain a permeable barrier material 21. A release liner 22 may be provided on the adhesive layer 20. The release liner layer 22 may be removed to expose the adhesive layer 20 before the transdermal patch 10 is applied to a patient's skin. In addition to the drug 15, the phase change material particles 16, and the material 18, the reservoir 14 may also contain other materials, such as permeation enhancers, thickeners, solvents, stabilizers, excipients, carriers and the like.

A similar embodiment is shown in FIG. 2. The difference between this embodiment and the one shown in FIG. 1 is the adhesive layer. In this embodiment, an adhesive layer 23 is along a perimeter of the transdermal patch 10 leaving an opening 24 exposing the permeable barrier material 21. When the release liner layer 22 is removed, both the adhesive layer 23 and the permeable barrier material 21 are in contact with the patient's skin. The rest of the description for the transdermal patch is the same as FIG. 1.

The backing layer 12 prevents the materials in the reservoir layer 14 from being released or transported through the top or side surfaces of the patch 10. This layer is preferably substantially impermeable with regard at least to the drug 15 used in the patch 10, if not to all of the materials in the reservoir 14 layer. Thus, the backing layer 12 also prevents the phase change material particles 16 or the material 18 from being released or transported through the top or side surfaces when the patch is unheated and also when heat is applied. The materials of which the backing layer 12 can be made include polymers such as polyethylene, polypropylene, polyvinylchloride, polyurethane, polyesters such as poly (ethylene phthalate), coated flexible fabrics such as paper or cloth, and foils such as laminates of polymer films with metallic foils such as aluminum foil. The backing layer 12 can be produced or provided in any thickness appropriate to providing the desired barrier and support functions. For example, a suitable thickness may be in a range from 10 to 200 microns. The baking layer may also be painted or coated to enhance its absorption of IR radiation.

The backing layer 12 and adhesive layer 20 must be able to position and retain the contents of the reservoir 14. The adhesive layer 20 must also be permeable to the drug 15 and to the material 18 when the material 18 is released from the phase change material 16, such that the drug 15 and the suspended material 18 diffuse osmotically through the adhesive layer 20. The adhesive layer 20 may be made from acrylics, synthetic rubbers, silicone or other suitable materials. The backing layer 12 may extend over the sides of, and be contiguous with, the adhesive layer 20, fully encapsulating the reservoir 14, as shown in FIG. 1.

The drug 15 may include one or more of many pharmacological agents or narcotic analgesics such as fentanyl, sufentanil, alfentanyl, remifentanil, morphine or other drugs known in the art suitable for use in a transdermal patch or any drugs that can be treated with naloxone or nalmefene. The drug 15 should be permeable through the adhesive layer 20, the permeable barrier material 21, and also permeable through the skin to which the transdermal patch is applied. The drug may be contained in a gel, cream, paste, slurry, or other suitable pharmaceutical carrier.

The phase change material, preferably in the form of particles 16, includes, holds, suspends, or contains the material 18. The phase change material 16 also acts as a barrier between the drug 15 and the suspended material 18 and must be impermeable to both of them. At room temperature, the phase change material is in a solid phase. With the addition of heat, the phase change material becomes liquid, releasing the material 18 into the reservoir 14. In one embodiment, the material 18 is mixed or blended into the phase change material creating the phase change material particles 16. In another embodiment, the material 18 may be blended with the phase change material to form a phase change material layer 16a, see FIGS. 3-7. In still another embodiment, the material 18 may be microencapsulated in the phase change material, rendering the particles 16 as microspheres. The microencapsulation process can be physical or chemical. The phase change material may be any material having the described characteristics and abilities. For example, high molecular weight alkanes (waxes) that melt at high temperatures, such as paraffin wax, may be utilized and are available with melting points or temperatures between 21.9° C. and 61.2° C. Other suitable materials include polylactones.

It may be desirable that the phase change material comprise one material having one melting point or temperature, with one material suspended therein. In other, more functionally rich applications, there may be multiple phase change materials having one or more melting points or temperatures and/or one or more materials suspended therein. The melting points or temperatures of the phase change materials may be tailored to initiate release of the suspended material at a specific phase change material melting point or multiple melting points.

The choice of material 18 depends on its intended purpose or role. One purpose of the material 18 may be to prevent unauthorized extraction and use of an analgesic drug from the patch 10. In this role, the material 18 is an antagonist to the drug 15, and those skilled in the art will appreciate that there must be an amount of the antagonist contained in the phase change material which is effective to counteract or neutralize the drug 15. In this configuration, when the transdermal patch 10 with phase change material is placed in boiling water to extract the drug 15, the heat would cause the phase change material to transition to a liquid, thereby releasing the antagonist material to counteract or neutralize the drug 15. As an example, presume the drug 15 is morphine. Accordingly, the material 18 is or includes a compound selected from the group of compounds that are antagonistic to morphine, which includes naloxone hydrochloride (Narcan) and nalmefene (Revex).

In another embodiment of this invention, the patch 10 may be used to assist a patient with a burst of an analgesic drug to treat breakthrough pain. Here, the material 18 may be identical to the drug 15 but in a more concentrated form than the drug 15, or it may comprise another analgesic drug that is more potent or effective for breakthrough pain. In use, the transdermal patch 10 is placed on the patient that is experiencing pain with a normal dosage of analgesic drug 15. When the patient experiences breakthrough pain heat is applied to the transdermal patch 10. In this case, heat may be applied by means of any one of a plurality of heat producing means including, without limitation, a resistance heater, a heating pad, a radiator, a concentrated beam of energy, or any equivalent means. When the melting point or temperature of the phase change material is reached, the phase change material transitions to liquid form, and the material 18 is delivered transdermally by the patch 10.

FIG. 3 illustrates another embodiment of this invention. The patch 10a is comprised of a backing layer 12, a reservoir 14a with a drug layer 15a and a phase change material layer 16a with a material 18 suspended or held in the phase change material while the phase change material is in its solid state, an adhesive layer 20a and a release liner 22. One difference between the patch 10 of FIG. 1 and the patch 10a of FIG. 3 is that in transdermal patch 10, the phase change material 16 is dispersed in the drug 15 while in transdermal patch 10a, the phase change material 16a is maintained or disposed separately from, but adjacent to, the drug 15a.

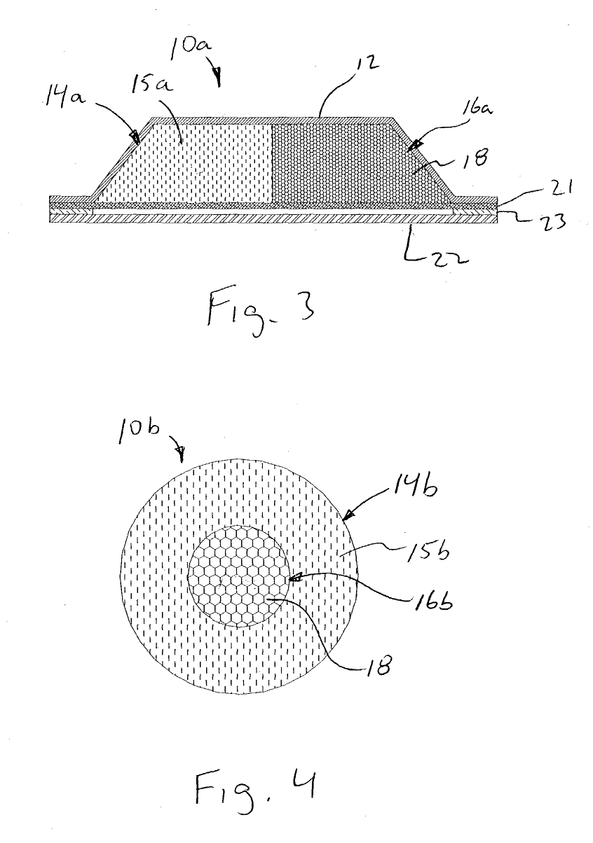

FIG. 4 is a sectional view looking down and illustrates another embodiment of this invention similar to FIG. 3, the difference being in the reservoir and phase change material layers. The patch 10b has a circular shape with a reservoir 14b with a drug layer 15b surrounding a phase change material layer 16b with a material 18 suspended or held in the phase change material while the phase change material is in its solid state.

FIG. 5 is a sectional view looking down and illustrates another embodiment of this invention similar to FIG. 3, the difference being in the reservoir and phase change material layers. The patch 10c has a circular shape, with a pie shape portion being a reservoir 14c with a drug layer 15c with a complementing pie shape portion being a phase change material layer 16c with a material 18 suspended or held in the phase change material while the phase change material is in its solid state.

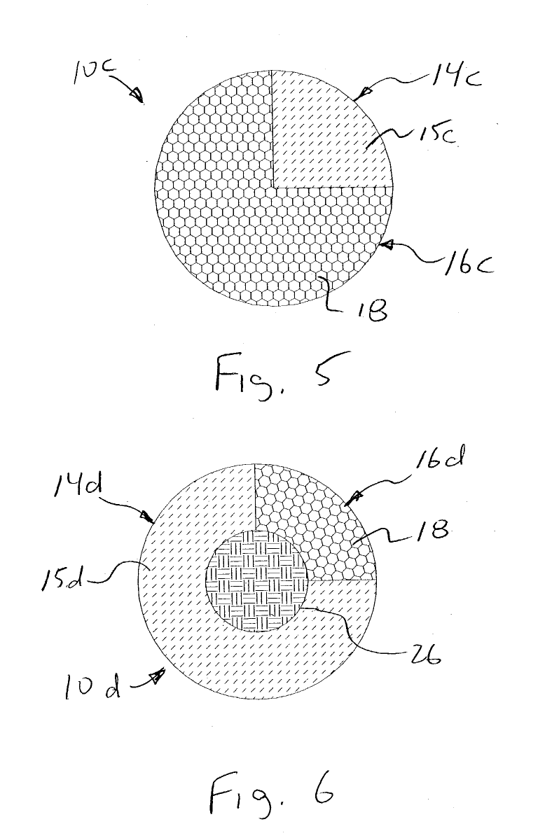

FIG. 6 is a sectional view looking down and illustrates another embodiment of this invention similar to FIGS. 3 and 5, the difference being in the reservoir and phase change material layers. The patch 10c has a circular shape, with a pie shape portion being a reservoir 14c with a drug layer 15c with a complementing pie shape portion being a phase change material layer 16c with a material 18 suspended or held in the phase change material while the phase change material is in its solid state. One difference between the patch 10c of FIG. 5 and the patch 10d of FIG. 6 is that near a center is another drug layer 26. This additional layer may be the same drug, a different dosage of the drug, another drug or another phase change material layer with another material suspended or held in the phase change material.

In still another embodiment, the reservoir of the patch may contain only the phase change material with a material suspended or held in the phase change material while the phase change material is in its solid state (i.e., no drug or drug layer in the reservoir). In this case, the patch may be used for specific applications or circumstances, such as the application of an analgesic drug. The analgesic drug would be mixed or suspended in the phase change material while the phase change material is in its solid state. For example, if a person needs a dose of pain reliever, a patch the phase change material with the suspended analgesic drug may be placed on the person's skin. Heat is then applied to the patch to liquefy the phase change material and release the suspended analgesic drug for transdermal introduction into the circulatory system of the patient.

EXAMPLE 1, ABUSE PREVENTION. In this example, a patch 10 contains an analgesic drug 15, such as morphine, for relieving pain in a patient. The dosage of morphine is selected to provide sustained and continuous delivery of the drug through the skin and into the circulatory system. There are cases where the transdermal patches are stolen to extract the analgesic drug 15 from the transdermal patch 10. One method of extracting the analgesic drug 15 is by boiling the transdermal patch 10 in water. To prevent this abuse, the transdermal patch 10 contains a morphine antagonist 18 suspended in a phase change material. When the transdermal patch 10 is heated in the boiling water, the phase change material changes to a liquid, releasing the antagonist 18, which then mixes with the morphine to counteract the morphine's effect. The amount of antagonist 18 in the phase change material should be enough to counteract the morphine. This antagonist may 15 also be released from the transdermal patch 10 if the patient tries to accelerate the dosing of the analgesic drug 15 by heating the transdermal patch 10, again preventing abuse.

EXAMPLE 2, BREAKTHROUGH PAIN RELIEF. In this example, a transdermal patch 10 contains an analgesic drug 15, such as morphine, for relieving pain in a patient. The dosage of morphine is selected to provide sustained and continuous delivery of the drug through the skin and into the circulatory system. In some instances, an additional amount of analgesic drug is needed by the patient for pain or for breakthrough pain episodes. To relieve breakthrough pain episodes, the transdermal patch 10 contains an additional dose of pain medication 18 suspended in a phase change material. To release the medication 18, heat is applied to the transdermal patch 10, changing the phase change material to a liquid, releasing the medication 18, which then migrates through the skin and into the circulatory system. In use, the release liner 22 is removed and the transdermal patch 10 is affixed to a patient's skin by the adhesive 20. The analgesic drug 15 is then delivered through the skin into the circulatory system of the patient, relieving pain. When a breakthrough pain episode occurs, sufficient heat is applied to the transdermal patch 10, for example by a resistance heater, or by a heating pad, to transition the phase change material from a solid to a liquid form, thereby releasing the additional dose of the medication 18. The additional dose may be the same as the drug 15, a more concentrated form of the drug 15, or a different drug altogether. Once the additional dose of the medication 18 is released from the phase change material, it migrates through the skin and into the circulatory system.

EXAMPLE 3, NICOTINE RELIEF. In this example, a patch 10 is a transdermal nicotine patch that contains a nicotine compound 15 for relieving nicotine urges in a patient trying to quit a nicotine habit. The dosage of the nicotine is selected to provide sustained and continuous delivery of the nicotine through the skin and into the circulatory system. In some instances, an additional amount of nicotine is needed by the patient to overcome an increased urge or craving. To relieve such a yen, the transdermal nicotine patch 10 may contain an additional dose of nicotine 18 suspended in a phase change material. To get this additional dose 18, heat is applied to the transdermal nicotine patch 10, changing the phase change material to a liquid, releasing the additional dose of nicotine 18, that is then delivered through the skin and into the circulatory system. In use, the release liner 22 is removed and the transdermal nicotine patch 10 is affixed to a patient's skin by the adhesive 20. The nicotine drug 15 is then delivered through the skin into the circulatory system of the patient, relieving the urge or craving associated with nicotine addiction. When a increased urge or craving episode occurs, sufficient heat is applied to the transdermal nicotine patch 10, for example with a heating pad, to change the phase change material from a solid to a liquid, releasing the additional dose of the nicotine 18. The additional dose of the nicotine 18 may be the same as the nicotine drug 15, a higher dose of the nicotine drug 15 and/or a different dose or drug completely. Once the additional dose of the nicotine 18 is released from the phase change material, it is delivered through the skin and into the circulatory system the same as the nicotine drug 15.

Many modifications and variations of the invention will be evident to those skilled in the art. It is understood that such variations may deviate from specific teachings of this description without departing from the essence of the invention.

We claim:

1. A transdermal device, comprising:
a backing layer of non-permeable material;
a reservoir adjacent to the backing layer;
a phase change material disposed in the reservoir in a solid form; and
an adhesive layer of permeable material adjacent to the reservoir opposite the backing layer.

2. The transdermal device of claim 1 further comprising a first drug disposed in the reservoir.

3. The transdermal device of claim 2, further comprising a second drug suspended in the phase change material when the phase change material is in solid form.

4. The transdermal device of claim 3, wherein the second drug is for being released from the phase change material when the phase change material is in liquid form.

5. The transdermal device of claim 3, wherein the first drug and the second drug are selected from the group of pharmacological agents or narcotic analgesics including fentanyl, sufentanil, alfentanyl, remifentanil, and morphine.

6. The transdermal device of claim 3, wherein the first drug is an analgesic drug of a first concentration and the second drug is an analgesic drug of a second concentration.

7. The transdermal device of claim 6, wherein the second concentration is higher than the first concentration.

8. The transdermal device of claim 2 further comprising an antagonist of the first drug suspended in the phase change material when the phase change material is in solid form.

9. The transdermal device of claim 8, wherein the antagonist is released from the phase change material when the phase change material is in liquid form.

10. The transdermal device of claim 9, wherein the first drug is an analgesic drug.

11. The transdermal device of claim 10, wherein the first drug is selected from the group of pharmacological agents or narcotic analgesics including fentanyl, sufentanil, alfentanyl, remifentanil, and morphine.

12. The transdermal device of claim 10, wherein the antagonist is selected from the group including naloxone hydrochloride (Narcan) and nalmefene (Revex).

13. The transdermal device of claim 2, wherein the phase change material is disposed in a phase change material layer and the first drug is in disposed in a first drug layer, the phase change material layer being positioned between the first drug layer and the backing layer.

14. The transdermal device of claim 2, wherein the phase change material is a plurality of phase change material particles dispersed within the first drug in the reservoir.

15. The transdermal device of claim 1 wherein the phase change material is selected from the group consisting of alkanes, paraffin waxes, poly lactones, and other suitable materials with phase change properties.

16. The transdermal device of claim 1, wherein the non-permeable material is selected from the group consisting of polyethylene, polypropylene, polyvinylchloride, polyurethane, polyesters such as poly(ethylene phthalate), coated flexible fabrics such as paper or cloth, and foils such as laminates of polymer films with metallic foils such as aluminum foil.

17. The transdermal device of claim 1 wherein the permeable adhesive material is selected from the group consisting of acrylics, synthetic rubbers, silicone and other suitable materials.

18. The transdermal device of claim 1 further comprising a release liner on the adhesive layer.

19. The transdermal device of claim 1 wherein a periphery of the backing layer is contiguous with a periphery of the adhesive layer.

20. A transdermal patch, comprising:
a backing layer of non-permeable material;
an adhesive layer of permeable material joined to the backing layer;
a reservoir between the backing layer and the adhesive layer;
a drug disposed in the reservoir;
a particulated phase change material dispersed in the drug;
a second material suspended in the phase change material when the phase change material has a solid form.

21. The transdermal patch of claim 20, wherein the second material is released from the phase change material into the reservoir when the phase change material is in liquid form.

22. The transdermal patch of claim 21, wherein the drug is an analgesic drug and the second material is an antagonist of the analgesic drug.

23. The transdermal patch of claim 22, wherein the drug is selected from the group of pharmacological agents or narcotic analgesics including fentanyl, sufentanil, alfentanyl, remifentanil, and morphine.

24. The transdermal patch of claim 22, wherein the antagonist is selected from the group including naloxone hydrochloride (Narcan) and nalmefene (Revex).

25. The transdermal patch of claim 21, wherein the drug is an analgesic drug of a first concentration and the second material includes an analgesic drug of a second concentration.

26. The transdermal patch of claim 25, wherein the second concentration is higher than the first concentration.

27. The transdermal patch of claim 21, wherein the drug and second material are selected from the group consisting of pharmacological agents or narcotic analgesics including fentanyl, sufentanil, alfentanyl, remifentanil, and morphine

28. The transdermal patch of claim 20, wherein the phase change material is selected from the group consisting of alkanes, paraffin waxes, poly lactones, and other suitable materials with phase change properties.

29. The transdermal patch of claim 20, wherein the non-permeable material is selected from the group consisting of polyethylene, polypropylene, polyvinylchloride, polyurethane, polyesters such as poly(ethylene phthalate), coated flexible fabrics such as paper or cloth, and foils such as laminates of polymer films with metallic foils such as aluminum foil.

30. The transdermal patch of claim 20, wherein the permeable adhesive layer is selected from the group consisting of acrylics, synthetic rubbers, silicone and other suitable materials.

31. The transdermal patch of claim 20 further comprising a release liner on the adhesive layer.

32. The transdermal patch of claim 20, wherein a periphery of the backing layer is contiguous with a periphery of the adhesive layer.

33. A method for drug delivery using a transdermal device which includes:
a first drug disposed in the device;
a phase change material disposed in the device; and
a second drug suspended in the phase change material when the phase change material has a solid form;
the method comprising:
attaching the transdermal device to skin for transdermal delivery of the first drug;
applying heat to the transdermal device to transition the phase change material to a liquid form; and
releasing the second drug from the phase change material for transdermal delivery of the second drug.

34. A method for drug delivery using a transdermal device which includes:
a drug in a first concentration disposed in the device;
a phase change material disposed in the device; and
a material including the drug in a second concentration higher than the first concentration suspended in the phase change material when the phase change material has a solid form;
the method comprising:
attaching the transdermal device to skin for transdermal delivery of the drug in the first concentration;
applying heat to the transdermal device to transition the phase change material to a liquid form; and
releasing the material from the phase change material for transdermal delivery of the drug in the second concentration.

35. A method for controlling use of a drug using a transdermal device which includes:
a drug disposed in the device for transdermal delivery;
a phase change material disposed in the device; and
a material including an antagonist of the drug suspended in the phase change material when the phase change material has a solid form;
the method comprising:
heating the transdermal device;
the heating causing the phase change material to transition to a liquid form; and,
releasing the material from the phase change material to neutralize the drug.
